(19) United States
(12) Patent Application Publication
(10) Pub. No.: US 2016/0279329 A1
(43) Pub. Date: Sep. 29, 2016

(54) SYSTEM AND METHOD FOR DRUG DELIVERY

(71) Applicant: Impreal Innovations Limited, London (GB)

(72) Inventors: Aldo FAISAL, London (GB); Cristobal LOWERY, London (GB)

(21) Appl. No.: 15/034,865

(22) PCT Filed: Nov. 7, 2014
(86) PCT No.: PCT/GB2014/053318

§ 371 Date: May 5, 2016

(30) Foreign Application Priority Data
Nov. 7, 2013 (GB) 1319681.1

(51) Int. Cl. A61M 5/172; G06F 19/00

(52) U.S. Cl. CPC A61M 5/1723; G06F 19/345; G06F 19/3468; A61M 2205/3334; A61M 2205/50; A61M 2202/0241

(57) ABSTRACT

A method and device for drug delivery is provided, in particular though not exclusively, for the administration of anaesthetic to a patient. A state associated with a patient is determined based on a value of at least one parameter associated with a condition of the patient. The state corresponds to a point in a state space comprising possible states and the state space is continuous. A reward function is provided for calculating a reward. The reward function comprises a function of state and action, wherein an action is associated with an amount of substance to be administered to a patient. The action corresponds to a point in an action space comprising all possible actions wherein the action space is continuous. A policy function is provided which defines an action to be taken as a function of state and the policy function is adjusted using reinforcement learning to maximize an expected accumulated reward.

The present disclosure relates to a system and method for drug delivery, in particular, though not exclusively, to the administration of anaesthetic to a patient.

The effective control of a patient's hypnotic state when under general anaesthesia is a challenging and important control problem. This is because insufficient dosages of the anaesthetic agent may cause patient awareness and agitation, but unnecessarily high dosages may have undesirable effects such as longer recovery times, not to mention cost implications.

Two techniques are currently used to control the infusion rate of the general anaesthetic agent.

The first consists of the anaesthetist adapting the infusion rate of the anaesthetic agent based on their judgement of the patient's current state, the patient's reaction to different infusion rates, and their expectation of future stimulus. The second, known as target-controlled infusion (TCI), assists the anaesthetist by using pharmacokinetic (PK) and pharmacodynamic (PD) models to estimate infusion rates necessary to achieve different patient states. Thus, in TCI it is only necessary to specify a desired concentration in the effect-site compartment (brain). However, TCI cannot fine-tune its response based on feedback, leading to it lacking the ability to account for inter-patient variability. Recent research has focused on investigating closed-loop control using a measure of a patient's hypnotic state, typically measured by the validated bispectral index (BIS). An example is a technique that targets a specific BIS value and uses the PK and PD models to estimate the necessary infusion rates to achieve the value. Another proposed example is a model-based controller that targets a specific BIS value, but it is based on proportional-integral-derivative (PID) control in order to calculate the infusion rate.

Although closed-loop algorithms have been proposed and tested with success, these algorithms heavily rely on models of a complex biological system that has a large amount of uncertainty and inter-patient variability. Moreover, the system is stochastic, non-linear and time dependant. As such, research suggests that the closed-loop control of a patient's depth of anaesthesia, or hypnotic state, yields itself better to the use of a reinforcement learner. However, known reinforcement learners for the control of general anaesthesia use a discrete state and action space, subjecting the system's generalisation capability to the curse of dimensionality. A priori discretisation also limits the available actions the reinforcement learner can take and, therefore, makes the algorithm sensitive to the discretisation levels and ranges. Moreover, known systems are trained using a typical patient, and do not learn during an operation. As such, such a reinforcement learner is not patient-adaptive.

The present disclosure describes a reinforcement learner that controls the dosage of a drug administered to a patient. In one embodiment, the reinforcement learner reduces the given dosage of anaesthetic, keeps the patient under tight hypnotic control, and also learns a patient-specific policy within an operation. The reinforcement learner aims to provide an automated solution to the control of anaesthesia, while leaving the ultimate decision with the anaesthetist.

In a first aspect there is provided a method for controlling the dose of a substance administered to a patient. The method comprises determining a state associated with the patient based on a value of at least one parameter associated with a condition of the patient, the state corresponding to a point in a state space comprising possible states wherein the state space is continuous. A reward function for calculating a reward is provided, the reward function comprising a function of state and action, wherein an action is associated with an amount of substance to be administered to the patient, the action corresponding to a point in an action space comprising possible actions wherein the action space is continuous. A policy function is provided which defines an action to be taken as a function of state and the policy function is adjusted using reinforcement learning to maximize an expected accumulated reward.

In some embodiments the method is carried out prior to administering the substance to the patient.

In some embodiments the method is carried out during administration of the substance to the patient.

In some embodiments the method is carried out both prior to and during administration of the substance to the patient.

An advantage of this method is that, for the policy function, only one action is learnt for a given state, as opposed to learning a probability of selecting each action in a given state, reducing the dimensionality by one and speeding up learning. Thus, the method has the advantage of finding real and continuous solutions, and it has the ability to form good generalisations from few data points. Further, since use of this method has the advantage of speeding up learning, the reinforcement learner can continue learning during an operation.

A further advantage of this method is that it is able to predict the consequences of actions. This enables a user to be prompted with actions recommended by the method and the consequences of such actions. It also enables manufacturers of a device carrying out this method to set safety features, such as detecting when the actual results stray from the predictions (anomaly detection) or preventing dangerous user interactions (for example, when in observation mode which is described below).

In some embodiments the method comprises a Continuous Actor-Critic Learning Automaton (CACLA). CACLA is an actor-critic setup that replaces the actor and the critic with function approximators in order to make them continuous.

An advantage of this method is that the critic is a value function, while some critics are Q-functions. If a Q-function had been used the input space would have an extra dimension, the action space. This extra dimension would slow down learning significantly due to the curse of dimensionality.

In some embodiments, the substance administered is an anaesthetic for example, propofol.

In some embodiments, the condition of the patient is associated with the depth of anaesthesia of the patient.

In some embodiments, the at least one parameter is related to a physiological output associated with the patient, for example, a measure using the bispectral index (BIS), a measure of the patient heart rate, or any other suitable measure as will be apparent to those skilled in the art.

In some embodiments, the state space is two dimensional, for example, the first dimension is a BIS error, wherein the BIS error is found by subtracting a desired BIS level from the BIS measurement associated with the patient, and the second dimension is the gradient of BIS. For example, the BIS gradient may be calculated by combining sensor readings with model predictions, as is described in more detail below and in detail in Annexes 1 and 2, and in brief in Annex 3 provided. Any other suitable method for calculating the BIS gradient may be used as will be apparent to those skilled in the art.

In some embodiments a state error is determined as comprising the difference between a desired state and the determined state, and wherein the reward function is arranged such that the dosage of substance administered to the patient and the state error are minimized as the expected accumulated reward is maximized.

In some embodiments, the reward function is a function of the square of the error in depth of anaesthesia (the difference between a desired depth of anaesthesia and a measured depth of anaesthesia) and the dosage of substance administered such that the reward function is maximised as both the square of the error and the dosage of substance administered are minimized. This is advantageous since the dosage of substance administered may be reduced while ensuring the desired depth of anaesthesia is maintained. This beneficial both to reduce the risk of overdose and the negative implications of this for the patient, and also to reduce cost since the amount of drug used is reduced.

For example, in some embodiments, the reward function, rₜ, may be given as:

rₜ=−[(BIS measurement associated with the patient−a desired BIS level)²]−0.02×Infusion Rate

In some embodiments, the action space comprises the infusion rate of the substance administered to the patient. The action may be expressed as an absolute infusion rate or as a relative infusion rate relative to a previous action, for example, the action at a previous time step, or a combination of absolute and relative infusion rates. This has the advantage of speeding up change of the substance dosage. In some embodiments, the method may comprise a combination of absolute and relative rates.

In some embodiments, the method operates relative to the weight or mass of the patient, and not the absolute quantities of the substance. This speeds up the adaptation of the substance dosage to individual patients. Alternatively or in addition, other physiological or anatomical parameters may be used in a similar manner, for example, patient height, gender, Body Mass Index, or other suitable parameters as will be apparent to those skilled in the art.

In some embodiments, the policy function is modelled using linear weighted regression using Gaussian basis functions. In other embodiments, any other suitable approximation technique may be used as will be apparent to those skilled in the art.

In some embodiments, the policy function is updated based on a temporal difference error.

In some embodiments the method updates the actor using the sign of the Temporal Difference (TD) error as opposed to its value, reinforcing an action if it has a positive TD error and making no change to the policy function for a negative TD error. This leads to the actor learning to optimise the chances of a positive outcome instead of increasing its expected value, and it can be argued that this speeds up convergence to good policies. This has the effect of reinforcing actions which maximise the reward.

In some embodiments, the action to be taken as defined by the policy function is displayed to a user, optionally together with a predicted consequence of carrying out the action.

In some embodiments a user is prompted to carry out an action as defined by the policy, for example, the prompt may be made via the display.

In some embodiments, the user is presented with a visual user interface that represents the progress of an operation. In embodiments where the state space is a one dimensional space, the visual user interface may be a two dimensional interface, for example, a first dimension may represent the state and the second dimension may represent the action, for example, the dose of substance.

In embodiments where the state space is a two dimensional space, the visual user interface may be a three dimensional interface, for example, the display may plot the dose of substance, the change in BIS measurement (time derivative) and the BIS measurement. Of course, in cases where are alternative measure to BIS measurements is used, this information may be displayed to the user. Similarly, the number of dimensions displayed by the visual user interface may depend on the number of dimensions of the state space.

In some embodiments the method can operate in ‘observer mode’. In this case, the reinforcement learning technique monitors an action made by a user, for example, the method may create a mathematical representation of the user. It assumes that the user chooses his or her actions based on the same input as the learner. Such a mode may be beneficial in identifying or preventing dangerous user interactions. This also enables tuning of the method, for example, for different types of operations with characteristic pain profiles.

In a further aspect, a reinforcement learning method for controlling the dose of a substance administered to a patient is provided, wherein the method is trained in two stages. In the first stage a general control policy is learnt. In the second stage a patient-specific control policy is learnt.

The method only needs to learn the general control policy once, which provides the default setting for the second patient specific stage of learning. Therefore, for each patient, only the second, patient-specific strategy needs to be learnt, making the process faster.

In some embodiments the general control policy is learnt based on simulated patient data. In some embodiments, the simulated patient data may be based on an average patient, for example, simulated using published data of a ‘typical’ or ‘average’ data. In other embodiments, the simulated patient data may be based on randomly selected patient data, for example, randomly selected simulated patients from a list of published patient data. Alternatively, the simulated patient data may be based on a simulated patient that replicates the behavior of a patient to be operated on, for example, following known pharmacokinetic (PK) and/or pharmacodynamic (PD) parameters proposed by known models and based on patient covariates (for example, age, gender, weight, and height).

In some embodiments, the general control policy is learnt based on the observer mode as described above. For instance, instead of training the reinforcement learner using simulated data as described above, the learner could be trained using real patients to follow an anaesthetist's approach. Optionally, following training using the observer mode, the method may be allowed to not only observe but to also act and as such improve its policy further.

In some embodiments, the patient-specific control policy is learnt during administration of the substance to the patient, for example, during an operation.

In some embodiments, the patient-specific control policy is learnt using simulated patient data, for example, as a means of testing the method.

In some embodiments, the method further comprises the features as outlined above.

In some embodiments, the average patient data and/or individual virtual patient specific data is provided using pharmacokinetic (PK) models, pharmacodynamics (PD) models, and/or published patient data.

In yet a further aspect, a device for controlling the dose of a substance administered to a patient is provided. The device comprising a dosing component configured to administer an amount of a substance to the patient, and a processor configured to carry out the method according to any of the steps outlined above.

In some embodiments, the device further comprises an evaluation component configured to determine the state associated with a patient.

In some embodiments, the device further comprises a display configured to provide information to a user.

In some embodiments the display provides information to a user regarding an action as defined by the policy function, a predicted consequence of carrying out the action and/or a prompt to carry out the action.

In some embodiments the device can operate in ‘observer mode’ as described above.

A specific embodiment is now described by way of example only and with reference to the accompanying drawings in which:

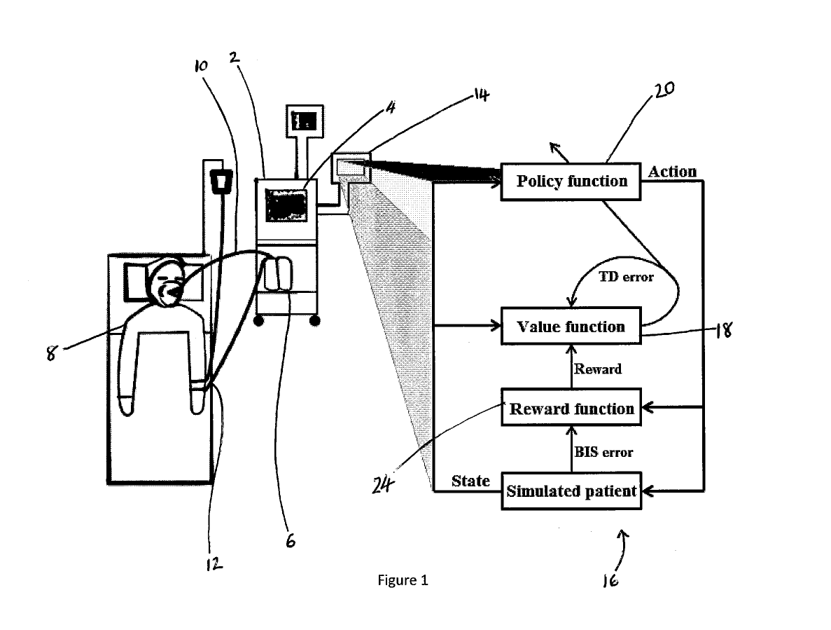

FIG. 1 shows a schematic view of a method according to this disclosure and a device for implementing the method; and

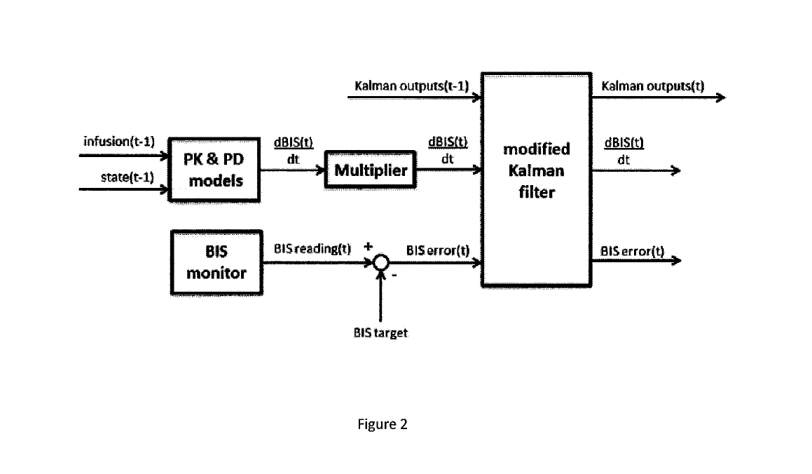

FIG. 2 shows a flow-diagram illustrating how the state space is constructed.

With reference to FIG. 1, a medical device 2 comprises a display 4 and a drug dispensing unit 6. The drug dispensing unit 6 is arranged to administer a drug to a patient 8. In some embodiments, the drug dispensing unit 6 is arranged to administer an anaesthetic, for example, propofol.

The drug dispensing unit 6 is arranged to administer the drug as a gas to be inhaled by the patient via a conduit 10. The drug dispensing unit 6 is also arranged to administer the drug intravenously via a second conduit 12. In some cases, in practice, a mixture of the two forms of administration is used. For example, where the drug administered is the anaesthetic, propofol, a dose of propofol is administered to the patient intravenously. Other drugs administered alongside propofol may be administered as a gas.

The drug dispensing unit 6 comprises a processing unit 14 having a processor. The processor is configured to carry out a continuous actor-critic learning automaton (CALCA) 16 reinforcement learning technique.

In overview, CALCA is a reinforcement machine learning technique composed of a value function 18 and a policy function 20. When given a state 22, the reinforcement learning agent acts to optimise a reward function 24. Both the value function and policy function are modelled using linear weighted regression using Gaussian basis functions, as will be described in more detail below, however, any suitable approximation technique may be used as will be apparent to those skilled in the art.

In the equations below, V(sₜ) represents the value function for a given state, s, and time, t, and finds the expected return. P(sₜ) represents the policy function at a given state and time, and finds the action which is expected to maximize the return. To update the weights corresponding to the two functions, equations (2) and (3) below are used, which are derived using gradient descent performed on a squared error function.

δ=rₜ₊₁+γ(sₜ₊₁)−V(sₜ) (1)

Wₖ(t+1)=Wₖ(t)+ηδφₖ(sₜ) (2)

Wₖ(t+1)=Wₖ(t)+η(αₜ−P(sₜ))φₖ(sₜ) (3)

In these equations. Wₖ(t) is the weight of the k^{th} Gaussian basis function at iteration t, and φₖ(sₜ) is the output of the k^{th} Gaussian basis function with input sₜ. The value function is updated at each iteration using (2), where δ represents the temporal difference (TD) error and η represents the learning rate. The TD error is defined in (1), where γ represents the discount rate, and rₜ₊₁ represents the reward received at time, t+1. The policy function was only updated when the TD error was positive so as to reinforce actions that increase the expected return. This was done using (3), where the action taken, a, consists of the action recommended by the policy function with an added Gaussian exploration term.

The state space used for both the value function and the policy function is two-dimensional. The first dimension was the BIS (bispectral index) error, found by subtracting the desired BIS level from the BIS reading found in the simulated patient. The second dimension was the gradient of the BIS reading with respect to time, found using the modeled patient system dynamics. In this embodiment, a measurement of BIS is used for the state space, in other embodiments, any physiological input may be used, for example, heart rate, or any other suitable measure as will be apparent to those skilled in the art.

The action space was the Propofol infusion rate, which was given a continuous range of values between 0-20 mg/min.

The reward function was formalized so as to minimize the squared BIS error and the dosage of Propofol, as: rₜ=−(BISError²)−0.02×InfusionRate. Alternatively, any strictly increasing function of BIS error may be used.

The reinforcement learning technique is described in more detail below and in detail in Annexes 1 and 2, and in brief in Annex 3 provided.

The reinforcement learner is trained by simulating virtual operations, which lasted for 4 hours and in which the learner is allowed to change its policy every 30 seconds. For each operation, the patient's state was initialized by assigning Propofol concentrations, C, to the three compartments in the PK model (described below), using uniform distributions (where U(a,b) is a uniform distribution with lower bound a and upper bound b): C1=U(0,50), C2=U(0,15), C3=U(0,2).

We introduced three elements in order to replicate BIS reading variability. The first was a noise term that varied at each time interval and followed a Gaussian distribution with mean 0 and standard deviation 1. The second was a constant value shift specific to each operation, assigned from a uniform distribution, U(−10,10). The third represented surgical stimulus, such as incision or use of retractors. The occurrence of the stimulus was modeled using a Poisson process with an average of 6 events per hour. Each stimulus event was modeled using U(1,3) to give its length in minutes, and U(1,20) to give a constant by which the BIS error is increased. As well as modeling the BIS reading errors, we provided that the desired BIS value for each operation varied uniformly in the range 40-60, for example, the desired BIS value may be 50. This pre-operative training phase for the reinforcement learner consisted of two episodes. The first learnt a general control strategy, and the second learnt a control policy that was specific to the patients' theoretical parameters. The reinforcement learner only needs to learn the general control strategy once, which provides the default setting for the second pre-operative stage of learning. Therefore, for each patient, only the second, patient-specific strategy needs to be learnt, making the process faster. In order to learn the first, general control strategy, we carried out 35 virtual operations on a default-simulated patient (male, 60 years old, 90 kg, and 175 cm) that followed the parameters specified in Schnider's PK model (described below and in the Annexes provided, in particular Annex 2). In the first 10 operations, the value function was learnt but the policy function was not. As a result, the infusion rate only consisted of a noise term, which followed a Gaussian distribution with mean 0 and standard deviation 5. In the next 10 operations, the reinforcement learner started taking actions as recommended by the policy function and with the same noise term. Here, the value of the discount rate used was 0.85, however, values approximately in the range 0.7 to 0.9 may be used, and the learning rate was set to 0.05. The final stage of learning performed 15 more operations with the same settings, with the exception of a reduced learning rate of 0.02.

The second learning episode adapted the first, general control policy to a patient-specific one. We did this by training the reinforcement learner for 15 virtual operations on simulated patients that followed the theoretical values corresponding to the actual age, gender, weight and height of the real patients as specified in Schnider's PK model. Once the pre-operative control policies were learnt, we ran them on simulated real patients to measure their performance. Here the setup was very similar to the virtual operations used in creating the pre-operative policies. However, one difference was that during the simulated real operations, the policy function could adapt its action every 5 seconds. This shorter time period was used to reflect the time frames in which BIS readings are received. The second difference was the method used to simulate the patients. To effectively measure the performance of the control strategy, it was necessary to simulate the patients as accurately as possible. However, there is significant variability between the behavior of real patients during an operation and that which is predicted by Schnider's PK model. As a result, in order to model the patients accurately, we used the data on nine patients taken from the research by Doufas et al (A. G. Doufas, M. Bakhshandeh, A. R. Bjorksten, S. L. Shafer and D. I. Sessler, “Induction speed is not a determinant of propofol”, Anesthesiology, vol. 101, no. 5, pp. 1112-21, 2004). This research used information from real operations to estimate the actual parameters of the patients, which are needed to model their individual system dynamics. To summarize, at the pre-operative learning stage we used theoretical patients based on Schnider's PK model, and to then simulate the reinforcement learner's behavior on real patients we used the data by Doufas et al.

In order to train the reinforcement learner, the expected change in BIS readings of a patient in response to the infusion rate of propofol is modeled. To do this a three stage calculation is used. The first stage was a PK model that was used to calculate plasma concentration at a given time based on the previous infusion rates of Propofol. Generally, Propofol concentrations are modeled using a mammillary three-compartmental model, composed of one compartment representing plasma concentration, and two peripheral compartments representing the effect of the body absorbing some of the Propofol and releasing it back into the veins. Propofol can flow between the compartments so that the concentration is equilibrated over time. To calculate the plasma concentration, we had to specify the volumes of the three compartments as well as the rate of Propofol elimination from them (rate constants). These parameters were patient-specific, and were approximated using the PK model proposed by Schnider, which is based on the patient's gender, age, weight and height. This technique is widely used and has been validated in human subjects. The second stage was a pharmacodynamic (PD) model that found the effect site concentration (in the brain) using plasma concentration. We modeled the PD by introducing a new compartment representing the effect site, connecting it to the central compartment of the PK model, and specifying the rate constant between the two compartments to a default value of 0.17 min⁻¹. The third stage used a three-layer function approximator (for example, an artificial neural network or sigmoid function (see Annex 2 for further detail)) to estimate the BIS reading from the effect site concentration.

Further detail on how patients may be modelled is provided in Annexes 1, 2 and 3 provided.

The CACLA technique is trained in a two-stage training phase. In the first stage, a general control strategy is learnt, and in the second a control policy specific to a patient's theoretical parameters is learnt. The reinforcement learner only needs to learn the general control strategy once, which provides the default setting for the second pre-operative stage of learning. Therefore, for each patient, only the second, patient-specific strategy need to be learnt, making the process faster and trainable during application to the patient.

The display 4 is used to provide the user with information regarding the potential consequences of following particular actions. The display 4 may also present to the user with a 3D visual interface that represents the progress of the operation in terms of the dose of propofol, change in BIS (the time derivative) and the BIS measurement itself. The display 4 may also provide a prompt to the user of an action to take.

Further detail regarding the embodiments described will now be provided below.

### Reinforcement Learning Framework

In choosing our reinforcement learning framework we considered our specific application and what we wanted to achieve. First, we considered that it was important to allow for actions to be kept in a continuous space. To then choose between actor-only and actor-critic, we had to consider whether the environment was changing quickly, in which case actor-only is preferred. Otherwise, an actor-critic framework is preferred as it provides for lower variance. Although the patient dynamics do change, we foresee that the evolution is moderate and partly accounted for by the second state space (dBIS/dt) and the modified PK-PD model. It was also felt that it would be important to learn a patient-adaptive strategy, which was a shortcoming of the paper we studied that uses reinforcement learning to control anaesthesia. In the paper, the policy was learnt in over 100 million iterations (10,000 operations), and, therefore, learnt too slowly to learn within an operation. For this reason, an option within the actor-critic framework is to use the CACLA technique, as it reduces the dimensionality of the actor and the critic by one dimension as compared to most actor-critic techniques. This dimensionality reduction is important in speeding up learning by several factors, and leads to the possibility to learn a patient-specific and patient-adaptive strategy.

Three important design choices are faced within the CACLA framework. The first is whether to reinforce all positive actions equally or to reinforce actions that improve the expected return more by a greater amount. If it is desired to stress different actions by different amounts, a technique known as CACLA+Var can be used. The second design choice is the exploration technique used. In this specific problem Gaussian exploration seemed most appropriate as the optimal action is more likely to be closer to the policies current estimate of the optimal action than further away, which is naturally accounted for by this form of exploration. Gaussian exploration has also been shown to be a better form of exploration than ε-soft policy for similar applications. The final design choice is which patient(s) to train the reinforcement learner on at the factory stage. The two options considered relied on using the data of patients 1 to 9 from Doufas et al. The first approach selected a patient for which we would test the reinforcement learner, and then used the mean Schnider PK values of the other eight patients and the mean PD values calculated for the patients using operation data. The second approach did not use the mean of the eight patients, but instead picked one patient at random for each simulated operation. Thus, we could compare the first approach to learning how to ride a bicycle by training on one typical bicycle, and the second approach by training on a series of eight different bicycles, thereby learning the structure of the problem. Both methods were tested, and the results of the policies learnt were comparable. Another important aspect in the design of the reinforcement learner was at what stage and at what rate the actor and the critic would learn. Given that the policy is evaluated by the critic, and the critic has lower variance, it is commonly accepted that it is best to learn the value function first or at a quicker pace. Thus, a common approach is to select a smaller learning rate for the critic than for the actor. An alternative is to first learn a value function for a predetermined policy. The predetermined policy chosen was to choose an infusion rate at each iteration by sampling from a uniform distribution U(0.025, 0.1) mg/minkg, a commonly used range of anaesthetists. Once this value function converged, which was estimated to occur after around five operations, a second stage of learning commenced. In the second stage, the policy function was used to select actions and was trained, resulting in an evolving actor and critic. Here the learning rates between the two functions were set to be equal. In this second stage, once convergence was observed, the Gaussian exploration term was reduced and so was the learning rate for both the policy and value function. At this stage a factory setting had been learnt, which is the policy that would be used when operating a patient for the first time. The third stage of learning occurred in the simulated real operations, where we set a low level of exploration. Here the policy evolved based on patient-specific feedback and learn an improved and patient-adaptive policy. Aside from the general framework, it was important to optimise a few heuristics of the reinforcement learner. The main heuristical elements were the length of each stage of learning, the learning rates used, and the noise chosen. In order to decide on values, each stage of learning was addressed in chronological order, and was optimised by testing the performance obtained when using a range of values of learning rates and exploration terms as well as waiting for convergence to determine how many operations should be used. Some of the heuristics that led to the best performance on the validation set are summarised in table 5.1. Two other parameter choices were the discount factor, γ, which was set to 0.85 and the time steps which were set to 30 seconds.

TABLE 5.1
Reinforcement learner's heuristic parameters.

| Stage | Operation numbers | Gaussian exploration $term [ \frac{mg}{minkg} ]$ | Actor learning rate (η) | Critic learning rate (η) |
|---|---|---|---|---|
| 1 | 1-5 | N/A | N/A | 0.03 |
| 2a | 6-12 | 0.05 | 0.03 | 0.03 |
| 2b | 13-18 | 0.03 | 0.02 | 0.02 |
| 3 | N/A | 0.02 | 0.01 | 0.01 |

### Actor and Critic Design

An important consideration in designing both the value and policy functions is what state space to use. One possible approach is to simply rely on the BIS monitor to provide a reading from which a BIS error can be calculated, seeing as the reinforcement learner has the target of minimising the square of the BIS error. However, this technique has the issue that the dynamics of a patient in response to Propofol infusion in two cases with equal BIS error can be very different. The same BIS error would be due to the effect-site compartment having similar concentrations of Propofol, and the difference in response to Propofol infusion would be due to different levels of Propofol having accumulated in the blood stream and other bodily tissues. Thus, for a given infusion rate (directly proportional to change in plasma concentration) and BIS level, the response in terms of BIS can vary significantly as the process is not memoryless. To capture this one idea would be to represent the state with the four compartmental concentrations from the PK-PD model. Although this solution would lead to a far more accurate representation, it introduces three new dimensions, significantly slowing down learning. Furthermore, there is no direct way of measuring these four concentrations. An alternative, which we use here, is to use a two-dimensional state space consisting of BIS error and d(BIS error)/dt (equivalent to dBIS/dt and we use the two interchangeably). This solution provides a far better representation of the state than just BIS error, it keeps the dimensionality of the state space low, and it can be estimated from BIS readings.

Given a two-dimensional input space, BIS error and dBIS/dt, it was necessary to design an appropriate function approximator for the critic and actor to map an input value to an expected return and optimal action, respectively. The function approximator chosen was LWR using Gaussian basis functions. In designing the LWR, a particular problem arises in that the input space is infinite in the dBIS/dt dimension and in the BIS error dimension some ranges of value are very rare. This is a problem for function approximators as we cannot learn the mapping with an infinite amount of basis functions, and the alternative of extrapolating predictions beyond the range of basis functions leads to poor predictions. Moreover, LWR performs poorly in predicting values outside the range in which there is a high enough density of training data due to over-fitting.

One solution that has been proposed is IVH, a technique that is used to stop the function approximator extrapolating results, removing the danger of poor predictions. However, this technique has no way of taking actions or evaluating policies outside this range, which is problematic. Thus, we have proposed limiting the input space our LWR uses for our actor and critic, and designing alternative rules for points outside the range. The first modification we applied in using LWR to estimate values was that of capping input values to the minimum or maximum acceptable levels in each dimension, and applying the LWR on these capped values. An exception to this rule was applied when the BIS reading was outside the range 40 to 60 (equivalent to BIS error −10 to 10). For these values, we believe it is necessary to warn the anaesthetist, allowing them to take over and perhaps benefit from any contextual knowledge that the reinforcement learner cannot observe. However, for the sake of our simulation and while the anaesthetist may have not reacted to the warning message, we feel it is appropriate to apply hard-coded values. In the case that BIS error is above 10, representing a too awake state, we apply a high, yet acceptable level of infusion, 0.25 mg/minkg. In the case of BIS errors below −10, no infusion is applied, allowing for the effect of the overdose to be reversed as quickly as possible. One option is to partition the input space that falls outside the acceptable range of values into a few regions, and learn an optimal value for each region. A second modification we apply is one that affects learning the weights of the function approximator. The rule applied is that any data point that falls outside the acceptable range of input values for that function approximator is discarded from the training dataset.

TABLE 5.2
Limits used on state spaces for two function approximators.

| Function | BIS | BIS | dBIS/ | dBIS/ |
|---|---|---|---|---|
| approximator | error min | error max | dt min | dt max |
| Actor | −10 | 10 | −1.2 | 1.2 |
| Critic | −15 | 15 | −1.8 | 1.8 |

In terms of the choice of limit for the actor, in one of the state space dimensions, the limit was naturally imposed by the acceptable range of BIS error. In the second dimension, the limits were decided by observing typical values in simulated operations and limiting the range to roughly three standard deviations, 1.5 on either side of the mean. Given this input space range, it was important to choose an input space range for the value function that successfully criticised the actor. For instance, if both the actor and the critic are limited to a maximum BIS error of 10, and the actor is in a state of BIS error equals 10, and it then takes two actions, in one case leading to a next state of BIS error equals 10 and in the second BIS error equals 11. All else equal, the critic would consider these two actions of equal value, as the BIS error of 11 would be capped to 10, before estimating its expected return. However, it is evident that the larger BIS error is worse. For this reason, it is important to balance making the critic's input space larger than that of the actor to minimise these situations and keeping it small enough to prevent poor approximations due to over-fitting. Another aspect in designing the function approximators for the actor and the critic is designing the output space. In the case of the value function, the output space corresponds to the expected return and is updated for each iteration where the state space is within an acceptable range. The TD error, δ, used to update the weights of the function approximator is given by equation 5.2. The reward function (equation 5.1) was formulated so as to penalise the squared BIS error, resulting in a larger relative penalisation for the bigger errors as compared to penalising just the absolute error term. Additionally, the equation penalises the action, which is the infusion rate as a proportion of the patient's weight, incentivising the agent to reduce the dosage. The reason for this design choice is that there are many adverse side effects associated to high dosages of Propofol. The choice of λ indicates the relative importance of infusion rate to squared BIS error. Here we chose a value of 10, which gives the infusion an importance of 12%, based on the average infusion rates and squared BIS errors observed in our simulated operations. We chose to give a lower importance to infusion rate than to squared BIS error, as under-weighting the importance of infusion has been shown to speed up learning. Moreover, by achieving tighter hypnotic control it is possible to set the target BIS level to a higher value and consequently reduce the infusion.

For the actor, the design of the output space is more complicated as it was necessary to ensure actions remained within a safe range. Moreover, we wanted to learn a policy that consisted of two terms, an absolute infusion rate and an infusion rate that is a multiple of the previous. The advantage of learning an absolute infusion rate is that it is memoryless and can, therefore, react more quickly to changing patient dynamics and surgical stimulus, amongst other factors, than the policy that is a multiple of the previous infusion rate. However, if we consider that we want to reach a steady state of BIS error equal to zero, it makes more sense to use a policy that is a multiple of the previous infusion rate. This is because if the infusion rate is too low to reach a sufficiently deep hypnotic state, then the infusion rate is increased, with the reverse effect when the infusion rate is too high. This can lead to the policy converging to an infusion rate that keeps the system stable around a BIS error equal to zero under stable patient conditions. Formally, the infusion rate at iteration k, uₖ [mg/min], output by the actor, was given as the combination of two policies leading to action₁ [mg/min] and action₂, the ratio of influence each equation has, ratio₁, patient i's weight, weightᵢ [kg], the previous infusion rate, uₖ₋₁ [mg/min], and a Gaussian distributed noise term with standard deviation, σ [mg/minkg] (equation 5.3). Action₁ corresponds to the absolute policy calculated using equation 5.5 and action₂ corresponds to the policy that is a multiple of the previous infusion rate calculated using equation 5.6. In order to learn the weights, w_{policy}, of the two function approximators used to output action₁ and action₂, the corresponding TD errors were calculated using equations 5.7 and 5.8. The TD error equations consist of two terms, the action performed and the action predicted. Finally, the infusion rate calculated using equation 5.3 was capped to a minimum value of 0.01 [mg/minkg] and maximum of 0.24 [mg/minkg], as calculated by dividing the infusion rate by the measured patient weight. The need to cap the infusion rate to a maximum below action^{max}₁ (set to 0.25) occurs as equation 5.7 is not solvable when the action taken corresponds to action^{max}₁, as the ln term becomes ln(0). The need to limit the minimum infusion rate above zero occurs as otherwise the second policy, that is a multiple of the previous infusion rate, will not be able to take an action in the next iteration.

$$\begin{matrix} {r}_{k + 1} = - {BISerror}_{k + 1}^{2} - λ {action}_{k} & ( 5.1 ) \\ δ = {r}_{k + 1} + γ \hat{V} ( {s}_{k + 1} ) - \hat{V} ( {s}_{k} ) & ( 5.2 ) \\ {u}_{k} = {action}_{1} ( {s}_{k} ) {weight}_{i} {ratio}_{1} + {action}_{2} ( {s}_{k} ) {u}_{k - 1} ( 1 - {ratio}_{1} ) + {weight}_{i} 𝒩 ( 0 , σ ) & ( 5.3 ) \\ {action}_{1} ( {s}_{k} ) = {action}_{1}^{max} sigmoid ( {w}_{policy 1}^{T} φ ( {s}_{k} ) ) & ( 5.4 ) \\ {action}_{1} ( {s}_{k} ) = \frac{{action}_{1}^{max}}{1 + exp ( - {w}_{policy 1}^{T} φ ( {s}_{k} ) )} & ( 5.5 ) \\ {action}_{2} ( {s}_{k} ) = max ( {action}_{2}^{min} , min ( {action}_{2}^{max} , exp ( {w}_{policy 2}^{T} φ ( {s}_{k} ) ) ) ) & ( 5.6 ) \\ {δ}_{{action}_{1}} = - ln ( \frac{{action}_{1}^{max}}{{u}_{k} / {weight}_{i}} - 1 ) - {w}_{policy 1}^{T} φ ( {s}_{k} ) & ( 5.7 ) \\ {δ}_{{action}_{2}} = max ( {action}_{2}^{min} , min ( {action}_{2}^{max} , ln ( \frac{{u}_{k}}{{u}_{k - 1}} ) ) ) - {w}_{policy 2}^{T} φ ( {s}_{k} ) & ( 5.8 )\end{matrix}$$

A few important design choices were made in equations 5.3 to 5.8. One of these was to express the output of action₁ using a sigmoid function (logistic function). This representation was used to ensure all output values were between zero and action^{max}₁. Another design choice was to use an exponential function with action₂. Using an exponential function ensures that the output multiple is never negative or zero, and naturally converts the output into a geometric rather than arithmetic form. A third design choice was of what minimum and maximum values to use to cap action₂ with. Too high absolute values of action₂ have the benefit of being reactive, but the issue of not helping the infusion rate to converge. Our results of several runs, in which both the policy and the resulting RMSE of the BIS error were looked over, led to the choice of values −1 and 1. Finally, it was important to decide on the influence of both of the policies on the final policy. In isolation, the first policy has a better average performance in terms of most medical metrics. However, imagine that one patient requires a significantly higher dosage to achieve the same hypnotic state as compared to the average patient on which the reinforcement learner has been trained. Then this patient will systematically not receive enough Propofol in the case of the first policy. The second policy would increase the infusion rate as necessary, not having the systematic shift in BIS.

As such, it was important to use both policies to benefit from each one's advantages, and find the right combination of influence between the two functions. Here we ran simulations and chose to set ratio₁ to 0.6, a level at which the RMSE of BIS error 2.89±0.07 (mean±standard error) was comparable to using the first policy in isolation 2.87±0.07, and at which we benefit from the influence of the second policy that is thought to be more robust.

### Linear Weighted Regression

The first choice in applying LWR was deciding what basis function to use. To make this choice we implemented both polynomial (quadratic and cubic) and Gaussian basis functions and tested their performance. Initially, it was expected that Gaussian basis functions would capture the function approximator more accurately, but at the cost of requiring more training data. The results showed that the polynomial basis functions had a few issues. When the function approximators were trained in batch form, the polynomials had worse predictive performance than the Gaussian basis functions. In the case of stochastic gradient descent, the predictive performance was very poor, which we believe was due to them being ill-conditioned. It may be possible to improve the performance of TD learning for the polynomial basis functions by using a Hessian matrix or Chebyshlev polynomials. Given the choice of Gaussian basis function for LWR, it was necessary to decide on several new parameters, namely the number of basis functions, their centres and their covariance matrices. One approach we followed to try to choose these parameters was, given a data set, to choose a number of basis functions approximately 100 times smaller than the number of data points, and to then apply stochastic gradient descent on all parameters, six per basis function (one weight, two centres, two standard deviations and one covariance). The results of this were negative, due to convergence issues. When watching the algorithm learn, it appeared that some of the six parameters per basis function learnt far quicker than others. This suggests that for this technique to be used successfully, it is necessary to apply different learning rates to different parameters. We chose, in one embodiment, to split up the learning task into a few stages. The first stage was to decide on the location of the basis functions (their centres). To do this we tried four different approaches to placing the basis functions, which included spreading them uniformly in each dimension, spreading them more densely at the centre of the grid than at the outside, applying Learning Vector Quantization (LVQ) on a set of data and using the learnt group centres, and finally applying MoG on a dataset. After using each technique to find the location of the basis functions, for each technique, various different covariance matrices were applied to the basis functions (using the same covariance for all basis functions) and then the covariance matrix which led to the lowest RMSE of BIS error in simulated operations was kept. In the case of MoG, the covariance matrices learnt were also tested. Although the MoG technique has the advantage of using the data to decide on locations, its learning clusters in two dimensions, while the data is in three dimensions. One approach was the one of hard-coded locations with the density of basis functions decreasing towards the outside of the grid. More precisely, these data points' coordinates in the BIS error direction were generated by evenly spacing out eight points starting at 0.1 and ending at 0.9. These points were then remapped, from x to y using equation 5.9, the inverse of a logistic function, thereby having the effect of increasing the density at the centre.

Finally, these new points were linearly mapped so that the minimum value ended up at −12 and the maximum at 12 (values slightly outside of the range for which the actor makes predictions). Then the same approach was applied to the dBIS/dt axis, using four points and a range of −1.7 to 1.7. The eight points found in one dimension were then combined with each of the four points found in the other direction, generating 32 coordinates.

$$y = - log ( \frac{1 - x}{x} ) \qquad ( 5.9 )$$

In order to decide on the covariance matrix of the basis functions, a few different ideas were considered and tested. One approach was to divide the region into 32 segments, one for each basis function, and assign each basis function the covariance of its segment. This technique was susceptible to systematically having too low or too high covariances. As such, we introduced a constant by which all of the basis functions' covariance matrices were multiplied, and tested a range of values for the constant to optimise its value. We found that this technique performed the least well. A second approach tested was using the covariance of all the training data, and applying this covariance, multiplied by a multiple to all basis functions. The results of this technique were better than the first. Finally, the third approach was to set the covariance to zero, and the standard deviation in each dimension equal to the range of values in that dimension divided by the number of Gaussians. Thus, for the actor in the BIS error dimension, in which there were eight points in the range of −12 to 12, the standard deviation was set to 3. These covariance matrices were then all multiplied by various multiples to pick the best value. This technique was the most successful, in terms of reducing the RMSE of predictions, and for this reason it was chosen. However, it would have been beneficial to introduce a technique that dynamically updated the covariance matrices to suit the evolving data densities. In terms of the multiplier chosen, it was found that the range 1 to 3 performed comparably well. When too large values are chosen, the function is too smooth and learning in one region affects learning in other regions, reducing the highly localised learning advantage of Gaussian basis functions. However, if the covariances are too small, the error not only increases, but there are disadvantages such as the value function becoming more bumpy, and forming various local minima that may mislead the policy function. Thus, to minimise the risk of either of these two issues, we chose a value of 2. In one embodiment, the covariance of the basis function may be varied to reflect the density of basis functions in the region, thereby increasing the covariance of basis functions towards the outside of the grid.

The last parameters to specify were the number of basis functions each dimension was divided into (in our case eight in the BIS error direction and four in dBIS/dt). In order to find the best choice, we started from a 2 by 2 grid, and increased each dimension individually, observing which one led to the greater performance gain. This was repeated until the performance, as measured by RMSE, reached a plateau. Our experiments also found that comparable performance could be obtained with a grid of 10 by 5, but we chose the fewer basis functions as this improves learning at the beginning and reduces the risk of over-fitting. The results suggest that it is more beneficial to segment the BIS error space than the dBIS/dt space, which is consistent with the fact that there is more variability in the output values in this dimension.

The choice of basis function centres, covariances, and the number used in each dimension, were determined by performing the described tests, applying the same rules to both the actor and the critic. This was done in order to reduce the dimensionality of the optimisation to a feasible level, but the functions outputs look quite different and this may be quite a crude generalisation. Thus, as a final stage, we attempted varying the multiple of the covariance matrix and changing the number of basis functions for each function approximator independently. The final design choice in LWR was of whether to use TD, LSTD, or batch regression to update the function approximators. The three techniques were implemented and tested in a few forms and the results led us to choose TD learning. Both LSTD and batch regression (equivalent to LSTD with a discount factor equal to 1), keep all previous data points and perform a matrix inversion (or Moore-Penrose pseudo inverse). This process leads to weights that reduce the function approximator's predictive squared error over the training set to the minimum possible value, in a sense leading to the optimal weights for our data set. However, there are two key issues with the two techniques. First, at the beginning of learning, when there are few data points, if we use basis functions, the weights learnt will be very poor due to over-fitting. One solution to this problem would be to begin learning with fewer basis functions and increase them in time. However, this solution would require various new heuristics for the number of basis functions, their locations and their standard deviations as well as how these parameters evolve in time. Moreover, even if we only started with very few basis functions, leading to a very poor fit, we would still not be able to get an acceptable fit initially with only a handful of data points. An alternative solution is to use a regularisation term to prevent over-fitting, but this would require the over-fitting parameter to evolve and be optimised for each iteration. Moreover, it would still be necessary to generate a large set of data points before the function learnt would be accurate. The second issue with LSTD and batch regression is that they give equal weighting to all data points, whilst the policy adapts quite quickly leading to both changing actors and critic, introducing a lag. This lag is very significant in our setup, due to the fact that we learn within an operation which has 480 iterations, of which typically around 200 iterations lead to policy data points. Thus, if we perform a regression on a dataset of 3000 data points (an advisable number for 32 basis functions) then the last operations dataset will constitute around 7% of the total data, and have a minimal effect on the learnt weights.

In contrast, TD learning performs gradient descent and, therefore, does not have the same issue of over-fitting or the same level of lag.

Further background regarding LWR and Temporal Difference and Least Squares Temporal Difference is provided in detail in Annex 2 provided.

### Kalman Filter

The reinforcement learner requires an estimate of BIS error, which can be obtained by subtracting BIS target from the value output by a BIS monitor. The monitor outputs values frequently, 1 Hz, but the output is noisy leading to a loss of precision in estimating a patient's true BIS state space. The reinforcement learner also requires a good estimate of dBIS/dt, which is hard to capture from the noisy BIS readings. Our in silico tests indicated that between two readings, a patient's change in true BIS state can be expected to account for approximately 1% of the change between the two readings, with noise accounting for the remaining 99%. Moreover, BIS shifts due to surgical stimulus would misleadingly indicate very large values of dBIS/dt. An alternative approach to estimating dBIS/dt would be to use a PK-PD model that follows a patient's theoretical parameters; however, this would not rely on a patient's true state but impose a predefined model. In order to make the best of both sources of information, we used a Kalman filter to estimate a patient's true BIS error and dBIS/dt, as shown in FIG. 2. The Kalman filter does not solely rely on BIS readings or model predictions, but instead fuses model predictions with sensor readings in a form that is optimised for Gaussian noise. Our Kalman was set up in an unconventional way as explained below.

dBIS(t)=(BIS(t)−BIS(t− 1/60))/( 1/60) (5.10)

In our configuration of the Kalman filter, the underlying system state that we are estimating is BIS error and the control variable is dBIS/dt. In order to estimate dBIS(t)/dt, the patient's theoretical PK-PD model is used to estimate BIS(t) and BIS(t−1), which are then entered into equation 5.10. This prediction is then multiplied by a multiplier that is learnt by the Kalman filter. Using the estimated value of dBIS(t)/dt, the BIS error(t) reading, and the posterior estimate of BIS error(t−1) and its covariance, the Kalman filter calculates a posterior estimate of BIS error(t). In our setup, in which the reinforcement learner changes its infusion rate once every 30 seconds, the Kalman filter is only called once every 30 seconds. For this reason, each time the Kalman filter is called, it has 30 BIS error readings and 30 dBIS/dt estimates, and it, therefore, performs 30 iterations, outputting only the results of the last iteration.

In our setup, we made a modification to the Kalman filter, as it assumes a constant value for B (a predefined linear constants used to describe the system (see equation 4.41 in Annex 2 for more detail)), whilst our data suggests that the PK-PD based estimates of dBIS/dt tend to be off by a constant factor. Thus, it was important to learn this factor, which we refer to as the multiplier, and adapt the estimates using it. Moreover, this factor can be seen to change throughout an operation, making it important for the multiplier to have the ability to change throughout the operation. The solution to this problem is to run three Kalman filters in parallel, each with its own value for B (0.91, 1 and 1.1), each time the Kalman filter function is called. The output of the three Kalman filters is then evaluated in order to select the best B and corresponding Kalman filter. This value of B is used to adjust the multiplier, by multiplying it by the selected value of B, and the selected Kalman filter is used to estimate the true BIS error. To estimate the true dBIS/dt value, the value of dBIS/dt predicted by the usual PK-PD models is multiplied by the learnt multiplier. In order to decide what the best value of B is at a given time, an RMSE was calculated between the 30 BIS errors based on readings and those output by each Kalman filter, leading to three RMSE values. If the control variable was systematically pushing the predictions up or down, the RMSE would increase, and as such a lower RMSE was taken to indicate a better choice of B. At first, there was concern that in the highly noisy environment it would be hard to use such a technique to distinguish better values of B, but this was tested and found to achieve the desired effect.

Our goal was to have as model-free an approach as possible; however, as mentioned previously, estimating dBIS(t)/dt purely from BIS readings with the level of noise our signal had would lead to poor results. Thus, it was necessary to include infusion rates to improve our model. However, the link between infusion rate and BIS value is very complex, and as such, including infusion rates in their raw format is of little use. For this reason, it was decided to convert infusion rates to estimates of dBIS/dt using the patient's PK-PD model. As such, it is important to understand what variability exists between a patient's expected reactions based on their theoretical parameters and their true reactions. One way of estimating this variability is to simulate a real patient using data from Doufas et al. and comparing the dBIS/dt estimated from the theoretical PK-PD patient to that of the real patient. This analysis led to the realisation that there is a high correlation between the values predicted and the true values, but that the ratio between the two predictions is typically far from one. It can also be observed that the ratio between the estimate and true values can change significantly throughout an operation. This suggested that our algorithm needed to estimate the ratio and to adapt this estimate as the operation progressed, and justified our design choice for the modified Kalman filter. The performance of the prediction modified by the learnt multiplier tends to be significantly better as judged by the coefficient of x being far closer to 1.

The last stage in configuring the Kalman filter required for three constants and two covariances to be specified (see equations 4.41 and 4.42 in Annex 2 for further detail). The constants F and H were set to 1, and B was set to 0.5 as dBIS/dt is output as a per minute rate, whilst the next BIS value that is being calculated for is half a minute into the future. The standard deviation of R was set to 1, as we assume that BIS readings have Gaussian noise with standard deviation 1. Finally, it was necessary to specify a value for Q, which we did by testing various values on the validation set of patients. To decide which value performed best, we considered the RMSE and analysed the output visually to find a good compromise between reducing the effect of noise and capturing large and quick shifts in BIS due to surgical stimulus. We set Q to 0.3, which for a simulated operation on patient 16 led to an RMSE of 0.46 between the Kalman estimate and the true value of BIS error, in comparison to an RMSE of 1.01 between the BIS reading and true BIS error. Here the true BIS error was the value calculated using our simulated patient, before applying measurement noise, and BIS readings were the true BIS values with added measurement noise. This configuration also performed well in terms of capturing BIS shift due to surgical stimulus.

Further background regarding Kalman Filters is provided in Annex 2 provided.

In use, a specific embodiment of the method is carried out, for example, according to the pseudo-code outlined in Annex 1 provided.

The system can also operate in observer mode. In this case, the learner monitors an action made by a user and may create a mathematical representation of the user. This assumes that the user chooses his or her actions based on the same input as the learner.

1. A method for controlling the dose of a substance administered to a patient, the method comprising:
determining a state associated with the patient based on a value of at least one parameter associated with a condition of the patient, the state corresponding to a point in a state space comprising possible states wherein the state space is continuous;
providing a reward function for calculating a reward, the reward function comprising a function of state and action, wherein an action is associated with an amount of substance to be administered to the patient, the action corresponding to a point in an action space comprising possible actions wherein the action space is continuous;
providing a policy function, which defines an action to be taken as a function of state; and
adjusting the policy function using reinforcement learning to maximize an expected accumulated reward.

2. A method according to claim 1, wherein the method is carried out prior to administering the substance to the patient.

3. A method according to claim 1, wherein the method is carried out during administration of the substance to the patient.

4. A method according to claim 1, wherein the method is carried out both prior to and during administration of the substance to the patient.

5. A method according to claim 1, wherein the method comprises a Continuous Actor-Critic Learning Automaton (CACLA).

6. A method according to claim 1, wherein a state error is determined as comprising the difference between a desired state and the determined state, and wherein the reward function is arranged such that the dosage of substance administered to the patient and the state error is minimized as the expected accumulated reward is maximised.

7. A method according to claim 1, wherein the substance is an anaesthetic.

8. A method according to claim 7, wherein the condition of the patient is associated with the depth of anaesthesia of the patient.

9. A method according to claim 1, wherein the at least one parameter is related to a physiological output associated with the patient.

10. A method according to claim 8, wherein the at least one parameter is a measure using the bispectral index (BIS).

11. A method according to claim 10, wherein the state space is two dimensional, the first dimension being a BIS error, wherein the BIS error is found by subtracting a desired BIS level from the BIS measurement associated with the patient, and the second dimension is the gradient of BIS.

12. A method according to claim 1, wherein the action space comprises the infusion rate of the substance.

13. A method according to claim 12, wherein the action may be expressed as an absolute infusion rate or as a relative infusion rate relative to a previous action, or as a combination of absolute and relative infusion rates.

14. A method according to claim 1, wherein the policy function is modelled using linear weighted regression using Gaussian basis functions.

15. A method according to claim 1, wherein the policy function is updated based on a temporal difference error.

16. A method according to claim 1, wherein the action to be taken as defined by the policy function is displayed to a user, optionally together with a predicted consequence of carrying out the action.

17. A method according to claim 1, wherein a user is prompted to carry out an action.

18. A reinforcement learning method for controlling the dose of a substance administered to a patient, wherein the method is trained in two stages, wherein:
a) in the first stage a general control policy is learnt;
b) in the second stage a patient-specific control policy is learnt.

19. A method according to claim 18, wherein the general control policy is learnt based on simulated patient data.

20. A method according to claim 19, wherein the simulated patient data is based on an average patient.

21. A method according to claim 19, wherein the simulated patient data may be based on randomly selected patient data.

22. A method according to claim 19, wherein the simulated patient data may be based on a simulated patient that replicates the behavior of a patient to be operated on.

23. A method according to claim 18, wherein the general control policy is learnt based on monitoring a series of actions made by a user.

24. A method according to claim 18, wherein the patient-specific control policy is learnt during administration of the substance to the patient.

25. A method according to claim 18, wherein the method further comprises the steps of claim 1.

26. A device for controlling the dose of a substance administered to a patient, the device comprising: a) a dosing component configured to administer an amount of a substance to the patient, b) a processor configured to carry out the method according to claim 1.

27. A device according to claim 26, wherein the device further comprises an evaluation component configured to determine the state associated with a patient.

28. A device according to claim 26, wherein the device further comprises a display configured to provide information to a user.

29. A device according to claim 28, wherein the display provides information to a user regarding an action as defined by the policy function, a predicted consequence of carrying out the action and/or prompt to carry out the action.
